(19) United States
(12) Patent Application Publication
(10) Pub. No.: US 2017/0135916 A1
(43) Pub. Date: May 18, 2017

(54) Dentifrice Compositions With Improved Fluoride Stability

(71) Applicant: The Procter & Gamble Company, Cincinnati, OH (US)

(72) Inventors: Swapna Basa, Beijing (CN); Ross Strand, Singapore (SG); James Albert Berta, West Chester, OH (US); Hongmei Yang, Beijing (CN)

(21) Appl. No.: 15/347,824

(22) Filed: Nov. 10, 2016

(30) Foreign Application Priority Data
Nov. 13, 2015 (WO) CN2015/094506

(51) Int. Cl. A61K 8/19; A61K 8/21; A61Q 11/00; A61K 8/02

(52) U.S. Cl. CPC A61K 8/19; A61K 8/0245; A61K 8/21; A61Q 11/00

(57) ABSTRACT

A dentifrice composition containing, water, calcium carbonate and a fluoride ion source where the calcium carbonate particles can have a D98 greater than 26.0 microns and the composition can have improved fluoride stability.

### FIELD OF THE INVENTION

The present invention relates to dentifrice compositions having high water and high carbonate levels and a fluoride ion source.

### BACKGROUND OF THE INVENTION

Dentifrice compositions are well known for dental and oral hygiene care. High water (e.g., >44 wt %) and high carbonate (e.g., >24 wt %) formulation chassis are a cost effective for many markets and consumers. However, this formulation chassis sometimes has fluoride ion stability issues that often exacerbated when there are high temperatures and/or long distribution times such as in some developing markets. Fluoride, and its associated benefits in dentifrice composition, is critical for a user's experience and product acceptance. There is a need to provide such dentifrice formulations having improved fluoride ion stability.

### SUMMARY OF THE INVENTION

The present invention is based, in part, on the surprising observation that in high water and high carbonate dentifrice formulations, calcium carbonate particles size has an important impact in fluoride stability. Furthermore, this fluoride ion stability effect is further enhanced at pH conditions that are greater than pH 8.0. Particle size distribution can be characterized by conventional D98, D90, D50, or D10 parameters.

Accordingly, an advantage of the present invention is improved soluble fluoride stability over time (in the high water high carbonate dentifrice formulation claimed herein).

One aspect of the invention provides for a dentifrice composition comprising: (a) 45% to 75%, by weight of the composition, of water; (b) 25% to 50%, by weight of the composition, of a calcium carbonate, wherein the calcium carbonate has a particle distribution size of D98 greater than 26.0 microns as measured by laser diffraction particle sizing per method ISO 13320-1-1999; (c) 0.0025% to 2%, by weight of the composition, of a fluoride ion source; and a pH greater than 8. Preferably the calcium carbonate has particle size distribution of D98 from 27 microns to 48 microns, more preferably from 30 to 47 microns, yet more preferably from 35 to 46 microns, yet still more preferably from 40 to 46 microns. More preferably the calcium carbonate has particle size distribution of D90 greater than 15.4 microns, preferably from 15.5 microns to 35 microns, more preferably from 16 to 34 microns, yet more preferably from 20 to 33 microns, yet still more preferably from 25 to 32 microns.

Another aspect of the invention provides for a method of treating tooth enamel comprising the step of brushing teeth with the aforementioned dentifrice composition.

While the specification concludes with claims that particularly point out and distinctly claim the invention, it is believed the present invention will be better understood from the following description.

### DETAILED DESCRIPTION OF THE INVENTION

### Definitions

The term “comprising” as used herein means that steps and ingredients other than those specifically mentioned can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “dentifrice” as used herein means paste, gel, powder, tablets, or liquid formulations, unless otherwise specified, that are used to clean the surfaces of the oral cavity. Preferably the dentifrice compositions of the present invention are single phase compositions. The term “teeth” as used herein refers to natural teeth as well as artificial teeth or dental prosthesis.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt %” herein.

As used herein, the articles including “a” and “an” when used in a claim, are understood to mean one or more of what is claimed or described.

As used herein, the terms “comprise”, “comprises”, “comprising”, “include”, “includes”, “including”, “contain”, “contains”, and “containing” are meant to be non-limiting, i.e., other steps and other sections which do not affect the end of result can be added. The above terms encompass the terms “consisting of” and “consisting essentially of”.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

### Calcium-Containing Abrasive

The compositions of the present invention comprise from 25% to 50% by weight of a calcium-containing abrasive, wherein the calcium-containing abrasive is calcium carbonate. Preferably the calcium carbonate is 27% to 47%, preferably 27% to 37%, more preferably from 28% to 34%, yet still more preferably from 29% to 33%, by weight of the composition. More preferably, the calcium-containing abrasive is selected from the group consisting of ground calcium carbonate, precipitated calcium carbonate, and combinations thereof. Fine ground natural chalk (FGNC) is one of the more preferred calcium-containing abrasives useful in the present invention. It is obtained from limestone or marble. FGNC may also be modified chemically or physically by coating during milling or after milling by heat treatment. Typical coating materials include magnesium stearate or oleate. The morphology of FGNC may also be modified during the milling process by using different milling techniques, for example, ball milling, air-classifier milling or spiral jet milling.

Particle size distribution of the calcium carbonate is a critical aspect of the present invention. The present invention is based, in part, on the surprising observation that in high water and high carbonate dentifrice formulations, calcium carbonate particles size has an important impact in fluoride stability. Particle size distribution (in microns) can be characterized by D98, D90, D50, and/or D10. The D50, the median, has been defined above as the diameter where half of the population lies below this value. Similarly, 98 percent of the distribution lies below the D98, 90 percent of the distribution lies below the D90, and 10 percent of the population lies below the D10.

Particle size characterization tools are well known including those based laser diffraction, dynamic light scattering, and dynamic image analysis. One suitable instrument is using a laser diffraction particle sizing instrument MASTERIES™ 2000 from MALDEN INSTRUMENTS using the methodology described in ISO 13320-1-1999.

One aspect of the invention provides a dentifrice composition wherein the calcium carbonate has a particle distribution size (microns) of D98 greater than 26.0, preferably from 27 to 48, more preferably from 30 to 47, yet more preferably from 35 to 46, yet still more preferably from 40 to 46, as measured in accordance to ISO 13320-1-1999. Preferably the calcium carbonate has particle size distribution (microns) of D90 greater than 15.4, preferably from 15.5 to 35, more preferably from 16 to 34, yet more preferably from 20 to 33, yet still more preferably from 25 to 32, as measured in accordance to ISO 13320-1-1999. More preferably, the calcium carbonate has particle size distribution (microns) of D50 greater than 6.0, preferably from 6.1 to 15, more preferably from 7 to 14, yet more preferably from 10 to 13, as measured in accordance to ISO 13320-1-1999. Yet more preferably, the calcium carbonate has particle size distribution (microns) of D10 greater than 0.7, preferably from 0.8 to 2.5, more preferably from 0.9 to 2.4, yet more preferably from 1 to 2.3, yet still more preferably from 1.5 to 2.1, as measured in accordance to ISO 13320-1-1999.

### Water

The dentifrice compositions of the present invention comprise herein from 45% to 75%, by weight of the composition, of water. Preferably the dentifrice composition comprises from 45% to 55%, more preferably from 46% to 54%, by weight of the composition, water. The water may be added to the formulation and/or may come into the composition from the inclusion of other ingredients. Preferably the water is USP water.

### Fluoride Ion Source

The compositions may include an effective amount of an anti-caries agent. In one embodiment, the anti-caries agent is a fluoride ion source. The fluoride ion may be present in an amount sufficient to give a fluoride ion concentration in the composition at 25° C., and/or in one embodiment can be used at levels of from 0.0025% to 5% by weight of the composition, alternatively from 0.005% to 2.0% by weight of the composition, to provide anti-caries effectiveness. Representative fluoride ion sources include: stannous fluoride, sodium fluoride, potassium fluoride, amine fluoride, sodium monofluorophosphate, and zinc fluoride. In one embodiment the dentifrice composition contains a fluoride source selected from stannous fluoride, sodium fluoride, and mixtures thereof. In one embodiment, the fluoride ion source is sodium monofluorophosphate, and wherein the composition comprises 0.0025% to 2%, by weight of the composition, of the sodium monofluorophosphate, alternatively from 0.5% to 1.5%, alternatively from 0.6% to 1.7%, alternatively combinations thereof. In another embodiment, the composition comprises from 0.0025% to 2%, by weight of the composition, of a fluoride ion source. In one example, the dentifrice compositions of the present invention may have a dual fluoride ion source, specifically sodium monofluorophosphate and an alkaline metal fluoride. Such an approach may provide an improvement in mean fluoride update.

pH

The pH of the dentifrice composition may be greater than pH 8.0, preferably from greater than pH 8 to pH 12. Preferably the pH is greater than 8.1, more preferably the pH is greater than pH 8.5, even more preferably the pH is greater than pH 9, alternatively the pH is from pH 9.0 to pH 10.5, alternatively from pH 9 to pH 10. The relatively high pH of the present inventive composition may help fluoride stability. Without wishing to be bound theory, at below pH 8 calcium ion may bind with the fluoride. Thus it is desirable to have the dentifrice composition have a greater than pH 8.0 to maximize the stability of the fluoride ion source. A method for assessing pH of dentifrice is described is provided in the analytical methods section provided below. For purposes of clarification, although the analytical method describes testing the dentifrice composition when freshly prepared, for purposes of claiming the present invention, the pH may be taken at anytime during the product's reasonable life cycle (including but not limited to the time the product is purchased from a store and brought to the consumer's home).

### pH Modifying Agent

The dentifrice compositions herein may include an effective amount of a pH modifying agent, alternatively wherein the pH modifying agent is a pH buffering agent. pH modifying agents, as used herein, refer to agents that can be used to adjust the pH of the dentifrice compositions to the above-identified pH range. pH modifying agents may include alkali metal hydroxides, ammonium hydroxide, organic ammonium compounds, carbonates, sesquicarbonates, borates, silicates, phosphates, imidazole, and mixtures thereof. Specific pH agents include monosodium phosphate (monobasic sodium phosphate or “MSP”), trisodium phosphate (sodium phosphate tribasic dodecahydrate or “TSP”), sodium benzoate, benzoic acid, sodium hydroxide, potassium hydroxide, imidazole, sodium gluconate, lactic acid, sodium lactate, citric acid, sodium citrate, phosphoric acid. In one embodiment, 0.01% to 3%, preferably from 0.1% to 1%, by weight of the composition, of TSP, and 0.001% to 2%, preferably from 0.01% to 0.3%, by weight of the composition, of monosodium phosphate is used. Without wishing to be bound by theory, TSP and monosodium phosphate may also have calcium ion chelating activity and therefore provide some monofluorophosphate stabilization (in those formulations containing monofluorophosphate).

### Thickening System

The dentifrice compositions of the present invention may comprise a thickening system. Preferably the dentifrice composition comprises from 0.5% to 4%, preferably from 0.8% to 3.5%, more preferably from 1% to 3%, yet still more preferably from 1.3% to 2.6%, by weight of the composition, of the thickening system. More preferably the thickening system comprises a thickening polymer, a thickening silica, or a combination thereof. Yet more preferably, when the thickening system comprises a thickening polymer, the thickening polymer is selected from a carboxymethyl cellulose, a linear sulfated polysaccharide, a natural gum, and combination thereof. Yet still more preferably, when the thickening system comprises a thickening polymer, the thickening polymer is selected from the group consisting of: (a) 0.01% to 3% of a carboxymethyl cellulose (“CMC”) by weight of the composition, preferably 0.1% to 2.5%, more preferably 0.2% to 1.5%, by weight of the composition, of CMC; (b) 0.01% to 2.5%, preferably 0.05% to 2%, more preferably 0.1% to 1.5%, by weight of the composition, of a linear sulfated polysaccharide, preferably wherein the linear sulfated polysaccharide is a carrageenan; (c) 0.01% to 7%, preferably 0.1% to 4%, more preferably from 0.1% to 2%, yet more preferably from 0.2% to 1.8%, by weight of the composition, of a natural gum; (d) combinations thereof. Preferably when the thickening system comprises a thickening silica, the thickening silica is from 0.01% to 10%, more preferably from 0.1% to 9%, yet more preferably 1% to 8% by weight of the composition.

Preferably the linear sulfated polysaccharide is a carrageenan (also known as carrageenin). Examples of carrageenan include Kappa-carrageenan, Iota-carrageenan, Lambda-carrageenan, and combinations thereof.

In one example the thickening silica is obtained from sodium silicate solution by destabilizing with acid as to yield very fine particles. One commercially available example is ZEODENT® branded silicas from Huber Engineered Materials (e.g., ZEODENT® 103, 124, 113 115, 163, 165, 167).

In one example the CMC is prepared from cellulose by treatment with alkali and monochloro-acetic acid or its sodium salt. Different varieties are commercially characterized by viscosity. One commercially available example is Aqualon™ branded CMC from Ashland Special Ingredients (e.g., Aqualon™ 7H3SF; Aqualon™ 9M3SF Aqualon™ TM9A; Aqualon™ TM12A).

Preferably a natural gum is selected from the group consisting of gum karaya, gum arabic (also known as acacia gum), gum tragacanth, xanthan gum, and combination thereof. More preferably the natural gum is xanthan gum. Xanthan gum is a polysaccharide secreted by the bacterium Xanthomonas camestris. Generally, xanthan gum is composed of a pentasaccharide repeat units, comprising glucose, mannose, and glucuronic acid in a molar ratio of 2:2:1, respectively. The chemical formula (of the monomer) is C₃₅H₄₉O₂₉. In one example, the xanthan gum is from CP Kelco Inc (Okmulgee, US).

### PEG

The compositions of the present invention may comprise polyethylene glycol (PEG), of various weight percentages of the composition as well as various ranges of average molecular weights. In one aspect of the invention, the compositions have from 0.01% to 8%, preferably from 0.1% to 5%, more preferably from 0.2% to 4.8%, yet more preferably from 0.3% to 4.2%, yet still more preferably from 0.5% to 4%, by weight of the composition, of PEG. In another aspect of the invention, the PEG is one having a range of average molecular weight from 100 Daltons to 1600 Daltons, preferably from 200 to 1000, alternatively from 400 to 800, alternatively from 500 to 700 Daltons, alternatively combinations thereof. PEG is a water soluble linear polymer formed by the addition reaction of ethylene oxide to an ethylene glycol equivalent having the general formula: H—(OCH₂CH₂)—OH. One supplier of PEG is Dow Chemical Company under the brandname of CARBOWAX™. Without wishing to be bound by theory, having some PEG in the dentifrice composition may help with physical stability.

### Anti-Calculus Agent

The dentifrice compositions may include an effective amount of an anti-calculus agent, which in one embodiment may be present from 0.05% to 50%, by weight of the composition, alternatively from 0.05% to 25%, alternatively from 0.1% to 15% by weight of the composition. Non-limiting examples include those described in US 2011/0104081 A1 at paragraph 64, and those described in US 2012/0014883 A1 at paragraphs 63 to 68, as well as the references cited therein. One example is a pyrophosphate salt as a source of pyrophosphate ion. In one embodiment, the composition comprises tetrasodium pyrophosphate (TSPP) or disodium pyrophosphate or combinations thereof, preferably 0.01% to 2%, more preferably from 0.1% to 1%, by weight of the composition, of the pyrophosphate salt. Without wishing to be bound by theory, TSPP may provide not only calcium chelating thereby mitigating plaque formation, but may also provide the additional benefit of monofluorophosphate stabilization (in those formulations containing monofluorophosphate).

### Surfactant

The dentifrice compositions herein may include a surfactant. The surfactant may be selected from anionic, nonionic, amphoteric, zwitterionic, cationic surfactants, or mixtures thereof. The composition may include a surfactant at a level of from 0.1% to 10%, from 0.025% to 9%, from 0.05% to 5%, from 0.1% to 2.5%, from 0.5% to 2%, or from 0.1% to 1% by weight of the total composition. Non-limiting examples of anionic surfactants may include those described at US 2012/0082630 A1at paragraphs 32, 33, 34, and 35. Non-limiting examples of zwitterionic or amphoteric surfactants may include those described at US 2012/0082630 A1 at paragraph 36; cationic surfactants may include those described at paragraphs 37 of the reference; and nonionic surfactants may include those described at paragraph 38 of the reference. In one embodiment the composition comprises 0.1% to 5%, preferably 0.1% to 3%, alternatively from 0.3% to 3%, alternatively from 1.2% to 2.4%, alternatively from 1.2% to 1.8%, alternatively from 1.5% to 1.8%, by weight of the composition, alternatively combinations thereof, of the anionic surfactant sodium lauryl sulfate (SLS).

### Low or Free Humectants

The compositions herein may be substantially free or free of humectants, alternatively contain low levels of humectants. The term “humectant,” for the purposes of present invention, include edible polyhydric alcohols such as glycerin, sorbitol, xylitol, butylene glycol, propylene glycol, and combinations thereof. In one embodiment, the humectant is a polyol, preferably wherein the polyol is selected from sorbitol, glycerin, and combinations thereof. In yet another embodiment, the humectant is sorbitol. In one embodiment, the composition comprises from 0% to less than 5%, by weight of the composition, of humectants, preferably from 0% to 4%, alternatively from 0% to 3%, alternatively from 0% to 2%, alternatively from 0% to 1%, by weight of th4 composition, of humectants. A potential advantage of having a dentifrice composition that is free or substantially free of humectants is, without wishing to be bound by theory, is those dentifrice compositions that are free of polyols (e.g., glycerin and sorbitol), or have a relatively low amount thereof, may provide better fluoride uptake compared to those compositions having the high levels of such polyols (or humectants for that matter). Preferably, the dentifrice compositions of the present invention comprise from 0% to 5%, preferably 0% to 3%, more preferably 0% to 1%, by weight of the composition, of glycerin, sorbitol, or combinations thereof; yet more preferably the composition is substantially free of both glycerin and sorbitol.

### Sweetener

The oral care compositions herein may include a sweetening agent. These sweetener agents may include saccharin, dextrose, sucrose, lactose, maltose, levulose, aspartame, sodium cyclamate, D-tryptophan, dihydrochalcones, acesulfame, sucralose, neotame, and mixtures thereof. Sweetening agents are generally used in oral compositions at levels of from 0.005% to 5%, by weight of the composition, alternatively 0.01% to 1%, alternatively from 0.1% to 0.5%, alternatively combinations thereof.

### Colorant

The compositions herein may include a colorant. Titanium dioxide is one example of a colorant. Titanium dioxide is a white powder which adds opacity to the compositions. Titanium dioxide generally can comprise from 0.25% to 5%, by weight of the composition.

### Flavorant

The compositions herein may include from 0.001% to about 5%, alternatively from 0.01% to 4%, alternatively from 0.1% to 3%, alternatively from 0.5% to 2%, alternatively 1% to 1.5%, alternatively 0.5% to 1%, by weight of the composition, alternatively combinations thereof, of a flavorant composition. The term flavorant composition is used in the broadest sense to include flavor ingredients, or sensates, or sensate agents, or combinations thereof. Flavor ingredients may include those described in US 2012/0082630 A1 at paragraph 39; and sensates and sensate ingredients may include those described at paragraphs 40-45, incorporated herein by reference. Excluded from the definition of flavorant composition is “sweetener” (as described above).

### Viscosity

A viscosity of 150000 cP to 850000 cP is a classic viscosity target range for a consumer acceptable dentifrice. The compositions of the present invention are preferably within this range. A method for assessing viscosity is described. The viscometer is Brookfield® viscometer, Model DV-I Prime with a Brookfield “Helipath” stand. The viscometer is placed on the Helipath stand and leveled via spirit levels. The E spindle is attached, and the viscometer is set to 2.5 RPM. Detach the spindle, zero the viscometer and install the E spindle. Then, lower the spindle until the crosspiece is partially submerged in the paste before starting the measurement. Simultaneously turn on the power switch on the viscometer and the helipath to start rotation of the spindle downward. Set a timer for 48 seconds and turn the timer on at the same time as the motor and helipath. Take a reading after the 48 seconds. The reading is in cP.

### Phase Stability

The term “phase stability” means visually (i.e., to the unaided eye) having no liquid separated from the oral care composition (e.g., toothpaste) body over a defined period of time under ambient conditions. In other words, a phase stable composition will resist syneresis. The compositions of the present invention are preferably phase stable for at least 6 months, more preferably 12 months or more.

### EXAMPLES

### Analytical Methods

The method for assessing soluble fluoride is described consistent with the China's National Standard Method GB8372-2008. Briefly, an ion-selective electrode (ISE) is used to test soluble fluoride in dentifrice. An example of a fluoride ion meter is SARTORIUS PP-50, but an equivalent may be used. The ion meter may be fitted with a fluoride-specific ion electrode with a single-junction reference electrode by Orion Research Inc., Cat. No. 9609BNWP, but an equivalent may be used. The sample is prepared by using a balance that is accurate to the 0.0001 gram (g). 20 g of dentifrice is weighed into a tarred 50 mL plastic beaker and then gradually 50 mL of deionized water is added, while a magnetic stir bar is stirring in the plastic beaker, until the dentifrice is a completely disperse solution. The entire solution is gently transferred to a 100 mL plastic volumetric flask as to avoid generating foam (so the volume can be measured accurately), and deionized water is added to reach a total volume 100 ml, and then the solution is shaken manually to form a slurry. The formed slurry is then transferred into 10 mL centrifuge tubes, and centrifued for 10 minutes at 15000 rotations-per-minute (RPM) (at 24149 g force) at ambient temperature. Thereafter 0.5 mL of supernatant is transferred into a 2 mL mini-centrifugal tube, and 0.7 mL of 4 mol/L HCl is added to the tub. Then the tub is capped, heated in a 50° C. waterbath for 10 minutes. Thereafter the contents of the tub are transferred to a 50 mL measuring flask. The following are also added to the flask: 0.7 mL of 4 mol/L NaOH to neutralize the solution; 5 mL of citrate buffer solution (described further below); deionzed water is added until a total volume of 50 mL is achieved in the flask; and then the sample solution is gently mixed. The aforementioned citrate buffer solution is prepared by dissolving 100 g of sodium citrate, 60 mL of glacial acetic acid, 60 g of NaCl, and 30 g of NaOH, all with water, adjusting the pH to 5.0-5.5, and diluting the citrate buffer solution with deionized water until a total volume of 1000 mL is achieved. Turning back to the sample solution, the entire 50 mL solution is transferred to a 50 mL plastic beaker and the fluoride level is assessed based on a fluoride standard curve using the fluoride ion meter and electrode described.

The standard fluoride curve (w/w %) is prepared by accurately measuring 0.5 mL, 1.0 mL, 1.5 mL, 2.0 mL, and 2.5 mL fluoride ion standard solutions (100 mg/kg) into five respective 50 mL plastic measuring flasks. 5 mL of citrate buffer solution (made as previously described above) into each respective flask, and then diluting each solution to the scale with deionized water. Thereafter, each solution is transferred into a 50 mL plastic beaker respectively, measuring potential E under magnetic agitation, recording potential values, and drawing E-logc (wherein “c” is a concentration) standard curve.

A method for assessing pH of dentifrice is described. pH is measured by a pH Meter with Automatic Temperature Compensating (ATC) probe. The pH Meter is capable of reading to 0.001 pH unit. The pH electrode may be selected from one of the following (i) Orion Ross Sure-Flow combination: Glass body—VWR #34104-834/Orion #8172BN or VWR#10010-772/Orion #8172BNWP; Epoxy body—VWR #34104-830/Orion #8165BN or VWR#10010-770/Orion #8165BNWP; Semi-micro, epoxy body—VWR #34104-837/Orion #8175BN or VWR#10010-774/Orion #3175BNWP; or (ii) Orion PerpHect combination: VWR #34104-843/Orion #8203BN semi-micro, glass body; or (iii) suitable equivalent. The automatic temperature compensating probe is Fisher Scientific, Cat #13-620-16.

A 25% by weight slurry of dentifrice is prepared with deionized water, and thereafter is centrifuged for 10 minutes at 15000 rotations-per-minute using a SORVALL RC 28S centrifuge and SS-34 rotor (or equivalent gravitational force, at 24149 g force). The pH is assessed in supernatant after one minute. After each pH assessment, the electrode is washed with deionized water. Any excess water is wiped with a laboratory grade tissue. When not in issue, the electrode is kept immersed in a pH 7 buffer solution or an appropriate electrode storage solution.

### Compositional Components

TABLE 1
Compositional components of inventive example 2
and comparative examples 1 and 3 are provided:

| Components: | Ex. 1 | Ex. 2 | Ex. 3 |
|---|---|---|---|
| (Wt %) | Comparative | Inventive | Comparative |
| CaCO₃ | 0 | 32.00 | 0 |
| ₍325 Mesh) | | | |
| CaCO₃ | 32.00 | 0 | 42.00 |
| (600 Mesh) | | | |
| Water | 55.52 | 55.52 | 45.52 |
| Sodium Mono- | 1.10 | 1.10 | 1.10 |
| fluorophosphate | | | |
| (“Na-MFP”) | | | |
| Sodium | 0.91 | 0.91 | 0.91 |
| Caboxy- | | | |
| methyl | | | |
| Cellulose | | | |
| Carrageenan | 1.41 | 1.41 | 1.41 |
| Thickener Silica | 2.62 | 2.62 | 2.62 |
| Sodium Lauryl | 4.00 | 4.00 | 4.00 |
| Sulfate | | | |
| Tetra Sodium | 0.42 | 0.42 | 0.42 |
| Pyrophosphate | | | |
| Flavor | 0.85 | 0.85 | 0.85 |
| Sodium Mono- | 0.08 | 0.08 | 0.08 |
| phosphate | | | |
| Sodium | 0.42 | 0.42 | 0.42 |
| Triphosphate | | | |
| Sodium | 0.58 | 0.58 | 0.58 |
| Saccharine | | | |
| Total: | 100 | 100 | 100 |
| Initial pH: | 9.39 | 9.39 | 9.34 |

Referring to Table 1, inventive example 2 differs from comparative examples 1 and 3 in at least one fundamental way, which is the relative size of the carbonate particles. The inventive composition notably contains a relatively larger calcium carbonate Mesh size 325 compared to the comparative examples 1 and 3 (having the smaller particle size of Mesh size 600). As between the comparative examples, example 3 contains more of the calcium carbonate (at 42 wt %) compared to example 1 (at 32 wt %). Particle size distribution is a more precise way of characterizing Mesh size.

### Data

Fluoride stability and pH change of the three examples are provided in Table 2. Examples 1 and 3 are comparative examples, while example 2 is an inventive composition. The compositional components of these examples are described in earlier Table 1.

TABLE 2a
Fluoride Stability Profile at 30° C. Two years

| | Examples: | | | | | |
|---|---|---|---|---|---|---|
| | Ex. 1 | | Ex. 2 | | Ex. 3 | |
| | (600M CaCO₃) | | (325M CaCO₃) | | (600M CaCO₃) | |
| | Comparative | | Inventive | | Comparative | |
| | Soluble | | Soluble | | Soluble | |
| | Fluoride | | Fluoride | | Fluoride | |
| Weeks: | (PPM) | pH | (PPM) | pH | (PPM) | pH |
| 0 | 1160 | 9.39 | 1200 | 9.39 | 1138 | 9.34 |
| 26 | 1132 | 8.86 | 1200 | 9.17 | 1000 | 8.92 |
| 52 | 912 | 8.92 | 1200 | 8.99 | 700 | 8.96 |
| 78 | 730 | 8.86 | 1279 | 8.81 | 494 | 8.97 |
| 104 | 592 | 8.91 | 1119 | 8.71 | 412 | 9.07 |

TABLE 2b
Fluoride Loss after Two years at 30° C.

| | Ex. 1 | Ex. 2 | Ex. 3 |
|---|---|---|---|
| | (32% 600M | (32% 325M | (42% 600M |
| | CaCO₃) | CaCO₃) | CaCO₃) |
| | Comparative | Inventive | Comparative |
| Total Soluble | 568 | 81 | 726 |
| Fluoride | | | |
| loss* (PPM) | | | |

*104 weeks

There are a number of observations that can be obtained from data of Tables 2a and 2b. Firstly, comparing the two comparative examples (Ex. 1 and 3) to each other, there is a greater drop in fluoride stability over the two years with example 3, i.e., the dentifrice composition containing more of the calcium carbonate. The only difference between examples 1 and 3 is the amount of calcium carbonate (and water). Both examples 1 and 3 have the relatively smaller calcium carbonate particles of Mesh size 600, but example 3 has a greater amount of the calcium carbonate (and less water) as compared to example 1. Over the course of two years, example 3 has soluble fluoride parts per million (PPM) loss of 726 while example 1 has a loss of 568 PPM. Accordingly, and given the greater soluble fluoride loss in example 3, there is a suggestion that a greater amount of calcium carbonate in the subject dentifrice chassis leads to greater fluoride stability loss. This underscored the need for a calcium carbonate type that can minimize or mitigate the loss of soluble fluoride over time in the higher water and high carbonate dentifrice formulation chassis described herein.

Comparing inventive example 2 to comparative example 1, the inventive dentifrice composition has significantly much lower loss of soluble fluoride over time. Over the course of two years, example 2 has soluble fluoride PPM loss of 726 while example 1 has only a loss of 568. The only difference between the examples 1 and 2 is the Mesh size (i.e., particle distribution) of the calcium carbonate particles. Inventive example 2 has the relatively larger particles of calcium carbonate of 325 Mesh size where as the comparative example 1 has the relatively smaller particles of calcium carbonate of 600 Mesh size. Similar results are observed comparing inventive example 2 and comparative example 3. Indeed comparative example 3 had the greatest amount of fluoride loss likely given that it has the most calcium carbonate and of the less desirable smaller Mesh sized calcium carbonate particles.

### Particle Size Distribution of Calcium Carbonate Particles

The particle size of the particulates in the CaCO3 is measured using a laser diffraction particle sizing instrument (Mastersizer™ 2000 from Malvern Instruments). The laser diffraction technique works by measuring the light scattered from particulates as they pass through a laser beam. Particulates scatter light at an angle that is directly related to their size. The Mastersizer™ 2000 uses the light scattering pattern associated with a sample to calculate particle size distributions. The instrument follows the recommendations of ISO 13320-1-1999. CaCO₃ raw material is dispersed into deionized water in Mastersizer™ 2000 sample beaker at 2000 rpm stirring speed. The dispersion is re-circulated between the beaker and the sampling cell of the particle sizing instrument where the particle size is measured. Particle size distribution parameters D98/D90/D50/D10 are obtained for each sample in the instrument standard software.

TABLE 3
Particle Size Parameters of Calcium Carbonate
particles of 325 Mesh and 600 Mesh sizes are
measured in accordance with ISO13320-1-1999.

| | |
|---|---|
| Particle Size | CaCO₃ Particle (microns) |

| Parameter | Size: 325M | Size: 600M |
|---|---|---|
| D10 | 2.058 | 0.7 (lower limit) |
| D50 | 12.224 | 3.0-6.0 |
| D90 | 31.711 | 7.4-15.4 |
| D98 | 45.021 | 26.0 (upper limit) |

As the data in Table 3 indicates, the particles size parameters of 325 Mesh calcium carbonate are much larger than those of 600 Mesh calcium carbonate. The 325 Mesh calcium carbonate can be obtained commercially from Guangxi Mantingfang Fine Chemical (Guangxi, China)

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An dentifrice composition comprising:
(a) 45% to 75%, by weight of the composition, of water;
(b) 25% to 50%, by weight of the composition, of a calcium carbonate,
wherein the calcium carbonate has a particle distribution size of D98 greater than 26.0 microns as measured by laser diffraction particle sizing per method ISO 13320-1-1999;
(c) 0.0025% to 2%, by weight of the composition, of a fluoride ion source; and a pH greater than 8.

2. The dentifrice composition according to claim 1, wherein the calcium carbonate has particle size distribution of D98 from 27 microns to 48 microns as measured by laser diffraction particle sizing per method ISO 13320-1-1999.

3. The dentifrice composition according to claim 2, wherein the calcium carbonate has particle size distribution of D98 from 35 microns to 46 microns as measured by laser diffraction particle sizing per method ISO 13320-1-1999.

4. The dentifrice composition according to claim 3, wherein the calcium carbonate has particle size distribution of D98 from 40 microns to 46 microns as measured by laser diffraction particle sizing per method ISO 13320-1-1999.

5. The dentifrice composition according to claim 1, wherein the calcium carbonate has particle size distribution of D90 greater than 15.4 microns as measured by laser diffraction particle sizing per method ISO 13320-1-1999.

6. The dentifrice composition according to claim 5, wherein the calcium carbonate has particle size distribution of D90 from 15.5 to 35 microns as measured by laser diffraction particle sizing per method ISO 13320-1-1999.

7. The dentifrice composition according to claim 6, wherein the calcium carbonate has particle size distribution of D90 from 20 to 33 microns as measured by laser diffraction particle sizing per method ISO 13320-1-1999.

8. The dentifrice composition according to claim 7, wherein the calcium carbonate has particle size distribution of D90 from 25 to 32 microns as measured by laser diffraction particle sizing per method ISO 13320-1-1999.

9. The dentifrice composition according to claim 1, wherein the calcium carbonate has particle size distribution of D50 greater than 6.0 microns as measured by laser diffraction particle sizing per method ISO 13320-1-1999.

10. The dentifrice composition according to claim 9, wherein the calcium carbonate has particle size distribution of D50 from 6.1 to 15 microns as measured by laser diffraction particle sizing per method ISO 13320-1-1999.

11. The dentifrice composition according to claim 1, wherein the calcium carbonate has particle size distribution of D10 is greater than 0.7 microns as measured by laser diffraction particle sizing per method ISO 13320-1-1999.

12. The dentifrice composition according to claim 11, wherein the calcium carbonate has particle size distribution of D10 from 1 microns to 2.3 microns as measured by laser diffraction particle sizing per method ISO 13320-1-1999.

13. The dentifrice composition according to claim 1, wherein the composition comprises from 27% to 37%, by weight of the composition, calcium carbonate.

14. The dentifrice composition according claim 1, wherein the composition comprises from 45% to 55%, by weight of the composition, water.

15. The dentifrice composition according to claim 1, further comprising a thickening system, wherein the thickening system is selected from the group consisting of a thickening polymer, a thickening silica, or combinations thereof.

16. The dentifrice composition according to claim 15, wherein the thickening system comprises a thickening polymer wherein the thickening polymer is selected from the group consisting of carboxymethyl cellulose, linear sulfated polysaccharide, natural gum, and combinations thereof.

17. The dentifrice composition according to claim 16, wherein the thickening polymer comprises from 0.01% to 3%, by weight of the composition, carboxymethyl cellulose.

18. The dentifrice composition according to claim 16, wherein the thickening polymer comprises from 0.01% to 2.5%, by weight of the composition, linear sulfated polysaccharide.

19. The dentifrice composition according to claim 18, wherein the linear sulfated polysaccharide comprises carrageenan.

20. The dentifrice composition according to claim 1, further comprising from 0.1% to 5%, by weight of the composition, polyethylene glycol.
